(19) United States
(12) Patent Application Publication
(10) Pub. No.: US 2013/0085145 A1
(43) Pub. Date: Apr. 4, 2013

(54) IMATINIB MESILATE PHARMACEUTICAL TABLET

(71) Applicant: Ravula Sayisiva Prasad, Hyderabad (IN)

(72) Inventor: Ravula Sayisiva Prasad, Hyderabad (IN)

(73) Assignee: SUVEN LIFE SCIENCES LIMITED, Hyderabad (IN)

(21) Appl. No.: 13/703,042

(22) PCT Filed: Jan. 10, 2011
(86) PCT No.: PCT/IN2011/000016

§ 371 Date: Dec. 10, 2012

(30) Foreign Application Priority Data
Jun. 21, 2010 (IN) 1732/CHE/2010

(51) Int. Cl. A61K 31/506

(52) U.S. Cl. CPC A61K 31/506
USPC 514/252.18

(57) ABSTRACT

The present invention provides a pharmaceutical tablet, comprising Imatinib in an amount of 50-75% w/w of the total tablet weight and comprising at least one pharmaceutically acceptable excipient without a binding agent and the process for preparation of the same.

### TECHNICAL FIELD

The present invention relates to the pharmaceutical tablet comprising 4-(4-methylpiperazin-1-ylmethyl)-N-[4-methyl-3-(4-pyridin-3-yl) pyrimidin-2-ylamino) phenyl]-benzamide or pharmaceutically acceptable salts thereof. The invention further discloses the process for the preparation of the same.

### BACKGROUND ART

Imatinib mesilate is chemically designated as 4-(4-methylpiperazin-1-ylmethyl)-N-[4-methyl-3-(4-pyridin-3-yl) pyrimidin-2-ylamino) phenyl]-benzamide methane sulfonate and its structural formula is

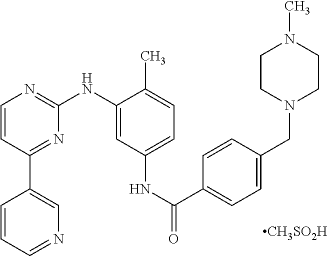

Imatinib mesilate is a white to off white to brownish or yellow tinged crystalline powder. Imatinib mesilate is soluble in aqueous buffers ≦pH 5.5 but is very slightly soluble to in soluble in neutral/alkaline aqueous buffers. In non-aqueous solvents, the drug substance is freely soluble to very slightly soluble in dimethyl sulfoxide, methanol and ethanol, but is insoluble in n-octanol, acetone and acetonitrile.

Imatinib is sold under the brand name GLIVEC (Imatinib as mesilate) which is marketed by Novartis pharmaceuticals. Glivec is available in tablets for oral administration in 100 mg and 400 mg strength. The inactive ingredients of Glivec are reported to be Tablet core: cellulose microcrystalline, crospovidone, hypromellose, magnesium stearate, silica colloidal anhydrous. Tablet coating: Iron oxide red (E172), Iron oxide yellow (E172), Macrogol, Talc and hypromellose.

Imatinib is a protein-tyrosine kinase inhibitor, especially useful in the treatment of various types of cancer and can also be used for the treatment of atherosclerosis, thrombosis, restenosis, or fibrosis. For the selected medicinal treatment as a protein-tyrosine kinase inhibitor for leukemia therapy Imatinib mesilate is administered in the high doses ranging from 100 mg to 800 mg especially from 400 mg to 800 mg of the active ingredient Imatinib. This means it is necessary to produce tablets with comparatively high active ingredient content preferably ranging from 25% by weight to 80% by weight, so that the tablet does not become too large.

EP1501485B1 describes tablets with a high active ingredient content of Imatinib for oral administration with atleast one binder e.g. microcrystalline cellulose or hydroxypropyl methyl cellulose produced by conventional wet granulation method. These tablets are expressed as being of small size and hence convenient to swallow despite the high dosage level of the active ingredient. However these tablets express high friability and poor abrasion resistance as well as limited flexibility in the amount of the excipients usable due to high drug load. In addition, the process of the manufacture of such tablets is difficult due to the poor flow of the final mixture and poor compressibility properties of such final mixture.

In the view of the nature of the active ingredient, the high dosage formulation related to the development the main objective is to develop a high dose formulation containing the active ingredient Imatinib mesilate by self granulation approach.

### SUMMARY OF THE INVENTION

In one embodiment the present invention provides a pharmaceutical tablet, comprising Imatinib in an amount of 50-75% w/w of the total tablet weight and comprising atleast one pharmaceutically acceptable excipient without a binding agent.

In the preferred embodiment the present invention provides a pharmaceutical tablet, comprising Imatinib in an amount of 50-75% w/w of the total tablet weight and comprising atleast one pharmaceutically acceptable excipients comprising the disintegrants e.g. crospovidone and sodium starch glycolate, at least one glidant, e.g. colloidal silicon dioxide, atleast one lubricant e.g. sodium stearyl fumarate, Talc and/or film coating.

In a preferred embodiment of the invention Imatinib is in the salt form of the Imatinib mesilate and the mesilate salt of Imatinib is in crystalline form e.g. alpha or beta crystal form preferably the alpha form.

One or more pharmaceutically acceptable excipients may be present in the tablets without a binding agent includes the disintegrants e.g. crospovidone and sodium starch glycolate, at least one glidant, e.g. colloidal silicon dioxide , atleast one lubricant e.g. sodium stearyl fumarate, Talc and/or film coating.

In the another embodiment the present invention provides the process for preparation of the pharmaceutical tablet comprising Imatinib in amount of 50-75% w/w of the total tablet weight and atleast one pharmaceutically acceptable excipient without a binding agent by the process of self granulation with the aid of a solvent.

In another embodiment the present invention relates to the process for the preparation of the pharmaceutical tablet comprising Imatinib in an amount of 50-75% w/w of the total tablet weight by 1) self granulating the Imatinib mesilate with the aid of a suitable solvent to obtain the granules 2) mixing the granules obtained in the step 1 with atleast one disintegrant, atleast one glidant and atleast one lubricant 3) compressing the mixture obtained in step 2 to form the tablets and 4) optionally coating the tablets with a film coating agent to form the film coated tablets.

### BRIEF DESCRIPTION OF THE DRAWINGS

The accompanied drawings which are incorporated herein and form part of the specification illustrate one or more embodiments of the present invention and, together with the description, further serve to explain the principles of the invention and to enable a person skilled in the pertinent art to make and use the invention.

FIG. 1 depicts the dissolution profile of Imatinib mesilate tablets of the present invention and in the Imatinib mesilate tablets sold under the trade name Glivec.

### DETAILED DESCRIPTION OF THE INVENTION

The specification discloses one or more embodiments that incorporate the features of the invention. The disclosed embodiment(s) merely exemplify the invention. The scope of the invention is not limited to the disclosed embodiment(s).The invention is defined by the claims appended hereto.

The present invention relates to the pharmaceutical tablet, comprising Imatinib or pharmaceutically acceptable salt thereof, preferably Imatinib mesilate, in an amount of 50-75% w/w of the total tablet weight and comprising atleast one pharmaceutically acceptable excipient without a binding agent.

The present invention further relates to the pharmaceutical tablet, comprising Imatinib or pharmaceutically acceptable salt thereof, preferably Imatinib mesilate, in an amount of 50-75% w/w of the total tablet weight and comprising atleast one pharmaceutically acceptable excipients comprising the disintegrants e.g. crospovidone and sodium starch glycolate, at least one glidant, e.g. colloidal silicon dioxide, atleast one lubricant e.g. sodium stearyl fumarate, Talc and/or film coating.

Suitable disintegrants according to the present invention include but are not restricted to crospovidone, sodium starch glycolate, croscarmellose sodium and Amberlite. Preferably crospovidone is used as the disintegrant.

As glidants one or more of the following may be used: Silica, colloidal silica, magnesium trisilicate. Preferably colloidal silicon dioxide is used as glidant.

As lubricants one or more of the following may be used magnesium stearate, calcium stearate, and Aluminium stearate and/or sodium stearyl fumarate and/or talc. Preferably Sodium stearyl fumarate and talc are used as lubricants.

According to the present invention the amount of disintegrant may vary within the range of from 10 to 30% in the weight based on the total weight of the tablet.

The amount of glidant may vary within the ranges of from 0.1% to 10% preferably from 1 to 3% in weight based on the total weight of the tablet.

The amount of lubricant may vary within the ranges of from 0.1 to 5% preferably 1 to 4% in weight based on the total weight of the tablet.

The amount of the film coating may vary from 1 to 10% preferably from 1 to 3% in weight based on the total weight of the tablet.

As used herein Self granulation means the drug Imatinib by itself forms the granules with the aid of a solvent.

The present invention relates to the process for the preparation of the pharmaceutical tablet comprising Imatinib in an amount of 50-75% w/w of the total tablet weight by 1) self granulating the Imatinib mesilate with the aid of a solvent to obtain the granules 2) mixing the granules obtained in the step 1 with atleast one disintegrant, atleast one glidant and atleast one lubricant 3) compressing the mixture obtained in step 2 to form the tablets and 4) optionally coating the tablets with a film coating agent to form the film coated tablets.

As solvents used for the self granulation the following may be used: purified water, methanol, ethanol, isopropyl alcohol and dimethyl sulfoxide. Preferably purified water is used as a solvent.

More specifically in one aspect of the present invention provides a process comprising:
1. Mixing the Imatinib mesilate in a high shear mixer
2. Adding purified water subjecting the mixture to wetting/kneading e.g in a high shear mixer, and drying e.g. Fluidized bed dryer or tray dryer.
3. Milling the granules obtained in step.2 in a granulator.
4. Adding the pharmaceutically acceptable excipients such as one or more disintegrants e.g. crospovidone, one or more glidants e.g. colloidal silicon dioxide and mixing with the granules of step no. 3 e.g. octagonal blender or double cone blender.
5. Adding the pharmaceutically acceptable lubricants such as one or more lubricants such as e.g. sodium stearyl fumarate and Talc and mixing e.g. octagonal blender or double cone blender.
6. Tabletting the mixture in step 5 by compression e.g. in conventional tablet press preferably a rotary machine.
7. Coating e.g. in a pan coater e.g. Neo cota (Automatic coating pan).

According to the invention, the coating process may be performed at low bed temperature, e.g. between 30 and 40° C., preferably between 32 and 39° C., most preferably at a temperature ranging from around 35 to around 38° C.

In a preferred embodiment of the invention tablets obtained by the compression method described above are round or oval. The edges of the tablets may be beveled or rounded. Most preferably, the tablets are ovaloid and/or round. The tablets according to the invention may be scored. The ovaloid tablet may be small in dimension e.g. 10 to 20 mm in length, preferably 15 to 20 mm, most preferably 16 to 19 nun; 5 to 10 mm in width, preferably 6.5 to 8 mm. The thickness of the tablet is from 4 to 8 mm, preferably 6 to 8 mm. Compression forces of between 10 to 20 kg/cm² are used to prepare the compressed tablet, preferably, 10 to 14 kg/cm². Preferably, the ovaloid tablet contains 400 mg of Imatinib. The round tablet may be of the following dimensions, e.g. 5 to 15 mm in diameter, preferably 7 to 10 mm, most preferably about 7.5 to 9 mm. The thickness of the tablet may be from 2 to 5 mm; preferably 2.5 to 4 mm. Compression forces of between 6 to 18 kg/cm² are used to prepare the compressed tablet, preferably, 8 to 14 kg/cm². Preferably, the round tablet contains 100 mg of Imatinib. Preferably the 100 mg tablet is a scored tablet; most preferably the tablet has a break score on one side.

The disintegration time of the tablet may be of about 20 min or less. Preferably, for the 100 mg Imatinib tablet, the disintegration time is ranging from about 2 to 10 min, preferably 4 to 10 min. For the 400 mg Imatinib tablet, the disintegration time is, preferably ranging from about 7 to 15 min, preferably 8 to 15 min.

In one embodiment, about 95% by weight Imatinib mesilate in the formulations of the present invention dissolves within 45 minutes, after being mixed in a USP Apparatus II (Paddle), under a mixing speed of 50 rpm at 37° C. in a dissolution medium of 900 ml of pH 1.2 medium.

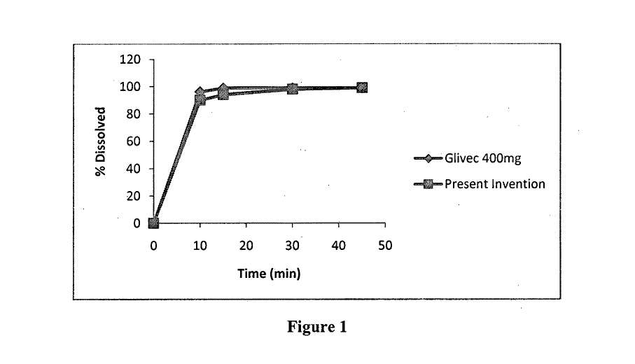

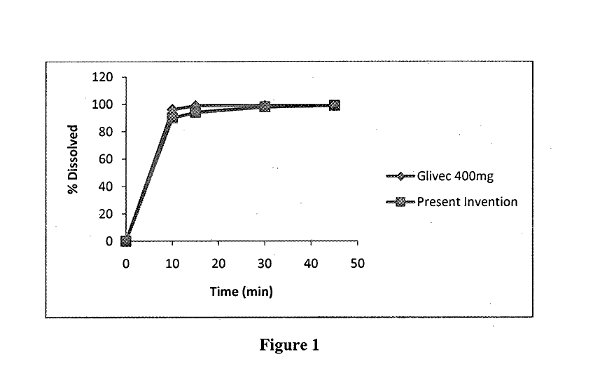

Dissolution studies demonstrate that the Imatinib mesilate tablets 400 mg of the present invention Example-2 exhibit a similar dissolution profile to the commercially available Glivec 400 mg tablets represented in FIG. 1.

### EXAMPLES

### Example-1

### Tablet Formulation 100 mg Tablet

Composition Per Dosage Form Unit

| | |
|---|---|
| Component | Composition per unit |

Intragranular portion

| | |
|---|---|
| Imatinib mesilate | 119.5 |
| Purified water | q.s |

Extra granular portion

| | |
|---|---|
| Crospovidone | 19.75 |
| Colloidal silicon dioxide | 2.5 |
| Talc | 1.5 |
| Sodium stearyl fumarate | 2.25 |

Tablet coating

| | |
|---|---|
| Insta coat | 2.3 |
| Total weight of the tablet | 147.8 |

Tablets of the 100 mg of the Imatinib according to the invention were prepared by wet granulation of Imatinib mesilate to form the granules and mixing the granules with crospovidone, colloidal silicon dioxide, talc and sodium stearyl fumarate, compressing the mixture to form the tablets and coating the resultant tablets with an aqueous dispersion of InstaCoat.

### Example-2

### Tablet Formulation 400 mg Tablet

Composition Per Dosage Form Unit

| | |
|---|---|
| Component | Composition per unit |

Intragranular portion

| | |
|---|---|
| Imatinib mesilate | 478 |
| Purified water | q.s |

Extra granular portion

| | |
|---|---|
| Crospovidone | 88 |
| Colloidal silicon dioxide | 6.2 |
| Talc | 6.4 |
| Sodium stearyl fumarate | 6 |

Tablet coating

| | |
|---|---|
| Insta coat | 6.4 |
| Total weight of the tablet | 591 |

Tablets of the 400 mg of the Imatinib according to the invention were prepared by wet granulation of Imatinib mesilate to form the granules and mixing the granules with crospovidone, colloidal silicon dioxide, talc and sodium stearyl fumarate, compressing the mixture to form the tablets and coating the resultant tablets with an aqueous dispersion of InstaCoat.

1. A process for the preparation of binder/binding agent free pharmaceutical tablets of Imatinib or its salts comprising of:
i) obtaining binder/binding agent free granules of Imatinib or its salts by self granulation of the Imatinib or its salts with the aid of a solvent;
ii). mixing the binder/binding agent free granules obtained in step (i) with atleast one pharmaceutically acceptable disintegrant and/or lubricant;
iii). compressing the mixture obtained in step (ii) to form binder/binding agent free pharmaceutical tablets of Imatinib or its salts.

2. A process as claimed in claim 1, wherein said solvent is selected from the group consisting of purified water, methanol, ethanol, and isopropyl alcohol.

3. A process as claimed in claim 2, wherein said solvent is purified water.

4. A process as claimed in claim 1, wherein said disintegrant is selected from the group consisting of cross linked polyvinyl pyrrolidone, sodium starch glycolate, croscarmellose sodium and amberlite.

5. A process as claimed in claim 4, wherein said disintegrant is cross linked polyvinyl pyrrolidone.

6. A process as claimed in claim 4, wherein said disintegrant is croscarmellose sodium.

7. A process as claimed in claim 1, wherein said lubricant is one or more selected from the group consisting of sodium stearyl fumarate, colloidal silicon dioxide and talc.

8. A process as claimed in claim 1, wherein said binder/binding agent free granules of Imatinib or its salts are obtained by self granulation of Imatinib or its salts only with the aid of said solvent.

9. A process as claimed in claim 1, wherein said pharmaceutically acceptable excipients are added only after the self granulation of said Imatinib or its salts.

10. A binder/binding agent free pharmaceutical tablet of Imatinib or its salts, wherein said tablet comprises binder/binding agent free granules of Imatinib in an amount of 50-75% w/w of total tablet weight.

11. A binder/binding agent free pharmaceutical tablet of Imatinib or its salts as claimed in claim 1 comprising binder/binding agent free granules of Imatinib in an amount of 50-75% w/w of total tablet weight and at least one pharmaceutically acceptable excipient selected from cross linked polyvinyl pyrrolidone, colloidal silicon dioxide, sodium stearyl fumarate and talc, wherein said pharmaceutical acceptable excipients are extra granular ingredients of the said tablet.
